(19) United States
(12) Patent Application Publication
(10) Pub. No.: US 2017/0209158 A1
(43) Pub. Date: Jul. 27, 2017

(54) PERCUTANEOUS LUMBAR AND THORACIC SPINE FACET FUSION

(71) Applicant: Seth K. Williams, Madison, WI (US)

(72) Inventor: Seth K. Williams, Madison, WI (US)

(21) Appl. No.: 15/407,625

(22) Filed: Jan. 17, 2017

Related U.S. Application Data
(60) Provisional application No. 62/286,334, filed on Jan. 23, 2016.

(51) Int. Cl. A61B 17/17; A61B 17/00; A61B 17/16

(52) U.S. Cl. CPC A61B 17/1757; A61B 17/1615; A61B 17/00234; A61F 2/4405

(57) ABSTRACT

A system for percutaneous facet joint fusion includes a guide wire, a drill guide, a sheath, and a drill bit. The guide wire is configured for placement through an incision and into a pedicle of a first vertebra, where the incision is posterior to the first vertebra. The drill guide includes an opening that is configured to receive a drill bit. The sheath is mounted to the drill guide and is configured to receive the guide wire such that the drill guide can be positioned adjacent to a facet joint formed by the first vertebra and a second vertebra. The drill bit is configured to create a void in the facet joint by way of the drill guide, where the void is configured to receive a bone graft or bone growth promoting material.

### CROSS-REFERENCE TO RELATED APPLICATIONS

The present application claims the priority benefit of U.S. Provisional Application No. 62/286,334 filed on Jan. 23, 2016, the entire disclosure of which is incorporated by reference herein.

### BACKGROUND

The lumbar spine refers to the lower back, and is where a human's spinal column curves inward toward the abdomen. The lumbar spine, which typically starts five to six inches below the shoulder blades, connects with the thoracic spine at the top and the sacral spine at the bottom. A human lumbar spine typically includes 5 vertebrae, although some individuals have 6 vertebrae in their lumbar spine. A typical single-level spinal segment consists of a disk that is located between a cephalad vertebra and a caudal vertebra. The cephalad and caudal vertebrae are connected to each other primarily through the disk anteriorly and a left and right facet joint posteriorly, and stabilized by ligaments and muscles and the intrinsic facet geometry. The facet joints are bony projections from the posterior vertebrae that are capped with cartilage, and that articulate with each other. The disk and facet joints allow some mobility between the vertebral segments, while in tandem with the ligaments and muscles, also provide spinal stability. There are several different conditions that can affect the lumbar and thoracic spine and cause pain, including disk problems such as degenerative disk disease, facet degeneration, scoliosis and other deformities, trauma, tumor, infection, spondylolisthesis, and stenosis.

Non-operative treatments such as physical therapy, medications, and exercise may be appropriate for some of these conditions, but sometimes it is determined that surgery is necessary. A lumbar and/or thoracic spine fusion can be used to help alleviate pain and/or stabilize the spine in some individuals who are suffering from the aforementioned ailments. A spinal fusion may be performed in a variety of manners, including a conventional extensile open surgical approach through a midline posterior incision. Alternatively, a minimally invasive approach may be utilized, using a series of smaller incisions in order to minimize the physiological burden of surgery. Spinal instrumentation systems, for example, pedicle screws and rods, are often used to stabilize the spine and improve the fusion rate, in both open and minimally invasive percutaneous procedures. Spinal instrumentation can be placed through a single extensile incision in conventional open surgery, or may be placed through a series of small incisions, which is commonly referred to as percutaneous pedicle screw instrumentation. The spinal instrumentation provides the stability component of the fusion operation, but a successful fusion depends on a biological component, which is new bone growth between vertebral segments. In order for this new bone growth to occur and achieve fusion, the biological environment must favor bone growth. This biological component of the fusion typically depends on placement of bone graft or a bone growth promoting material between vertebral segments, which stimulates new bone growth from one vertebra to the next and results in a spinal fusion. The biological component of existing minimally invasive fusion techniques relies on an interbody fusion, whereby the disk between the two vertebrae being fused is removed and replaced with structural and/or biological material (i.e., bone graft or a bone growth promoting material) to promote bone growth across the intervertebral space.

### SUMMARY

An illustrative technique and system for percutaneous lumbar and thoracic spine facet fusion includes a drill guide, a drill to denude the facet joint and create a void or cavity in the facet joint, and bone graft or other bone growth promoting material to fill the void or cavity and promote a fusion across the facet joint.

An illustrative system for percutaneous facet joint fusion includes a guide wire, a drill guide, a sheath, and a drill bit. The guide wire is configured for placement through an incision and into a pedicle of a first vertebra, where the incision is posterior to the first vertebra. The drill guide includes an opening that is configured to receive a drill bit. The sheath is mounted to the drill guide and is configured to receive the guide wire such that the drill guide can be positioned adjacent to a facet joint formed by the first vertebra and a second vertebra. The drill bit is configured to create a void in the facet joint by way of the drill guide, where the void is configured to receive a bone graft or bone growth promoting material.

An illustrative method for performing percutaneous facet joint fusion includes inserting a guide wire through an incision and into a pedicle of a first vertebra. The method also includes inserting the guide wire into a sheath of a drill guide. The method also includes sliding the drill guide and the sheath along the guide wire to position the drill guide adjacent to a facet joint formed by the first vertebra and a second vertebra. The method also includes placing a drill bit through an opening in the drill guide and drilling into the facet joint to create a void. The method further includes placing a bone graft or bone growth promoting material into the void.

Another illustrative method for performing percutaneous facet joint fusion includes forming an incision that is posterior to a first vertebra. The method also includes inserting a drill guide through the incision and positioning the drill guide such that the drill guide is adjacent to a facet joint formed by the first vertebra and a second vertebra. The method also includes placing a drill bit through an opening in the drill guide and drilling into the facet joint to create a void. The method further includes placing a bone graft or bone growth promoting material into the void.

The foregoing is a summary of the disclosure and thus by necessity contains simplifications, generalizations, and omissions of detail. Consequently, those skilled in the art will appreciate that the summary is illustrative only and is not intended to be in any way limiting. Other aspects, features, and advantages of the devices and/or processes described herein, as defined by the claims, will become apparent in the detailed description set forth herein and taken in conjunction with the accompanying drawings.

### BRIEF DESCRIPTION OF THE DRAWINGS

The present disclosure will become more readily apparent from the specific description and the following drawings, in which:

FIG. 1A depicts a drill guide, in accordance with an illustrative embodiment.

FIG. 1B depicts a drill guide with an attached sheath, which can slide over a guide wire that has been placed in a pedicle caudal to the facet to be fused, in accordance with an illustrative embodiment.

FIG. 2A depicts a lateral view of the relevant anatomy of a lumbar spine.

FIG. 2B depicts the drill guide docked on a facet joint in the lumbar spine, with the lumbar spine depicted in the lateral view, in accordance with an illustrative embodiment.

FIG. 3 depicts a lateral view of the lumbar spine, with a guide wire placed through a pedicle and docked in the vertebral body, in accordance with an illustrative embodiment.

FIG. 4 depicts the drill guide with attached sheath, with the guide wire depicted in FIG. 3 passing through the sheath and thereby guiding the drill guide onto the facet joint to be fused, in accordance with an illustrative embodiment.

FIG. 5A depicts a drill bit that can be used to drill across the facet joint as part of the facet fusion technique, in accordance with an illustrative embodiment.

FIG. 5B depicts the drill bit of FIG. 5A passing through the drill guide and drilling across the facet joint, in accordance with an illustrative embodiment.

FIG. 6 depicts the drill guide docked on the facet joint as shown in FIG. 4, after the drill bit has been removed, with a void created across the facet joint by the drill bit, in accordance with an illustrative embodiment. The void will subsequently be filled with bone graft or other bone growth promoting material to provide the biologic component of the fusion.

FIG. 7A depicts a posterior view of a cephalad vertebra and a caudal vertebra and their associated facet joint, in accordance with an illustrative embodiment.

FIG. 7B shows the cephalad and caudal vertebrae with the void created in the facet joint by the drill bit, in accordance with an illustrative embodiment.

FIG. 8 is a flow diagram depicting a process for performing a percutaneous lumbar and/or thoracic spine fusion, in accordance with an illustrative embodiment.

### DETAILED DESCRIPTION

Posterior lumbar and thoracic spinal fusions are typically performed through one or more incisions placed on the posterior aspect of the spine. A single incision can be made in the midline and then the soft tissues stripped off the posterior aspect of the spine to expose the transverse processes of two or more spinal levels. The transverse processes are decorticated and bone graft or bone growth promoting material placed between the transverse processes, performing what is referred to as a posterolateral fusion. An open posterior spinal fusion may also be performed by accessing the disk space, which involves navigating past the neural elements, removing the disk material, and placing bone graft or bone growth promoting material within the intervertebral space. Alternatively, for a minimally invasive fusion, multiple incisions can be made through the skin or fascia off the midline, the muscles split along natural planes rather than stripped, and the interbody space exposed and the interbody fusion performed in a similar manner to the open interbody fusion procedure. The minimally invasive technique also requires navigation past the neural elements. With minimally invasive techniques, a reliable method has not been developed to perform a fusion that does not utilize the interbody space.

Percutaneous pedicle screws may be placed posteriorly through small incisions, each incision accommodating one, two, or rarely three, pedicle screws. These screws are then connected to each other with rods, thus stabilizing the spine. One of the more common reasons to place the pedicle screws and rods is to stabilize the spine as part of a spinal fusion procedure. Interbody fusion across the disk space can be performed through an anterior, anterolateral, lateral, or posterior technique, and under these circumstances, posterior percutaneous pedicle screws can then be placed through separate small incisions, thus stabilizing the spine.

A drawback to traditional intervertebral fusion is that the disk space is located relatively deep within the human body, and access to the disk space is therefore sometimes risky or difficult. With a posterior surgical approach, the surgeon must navigate past the neural elements in order to access the disk space, and/or the muscles stripped off the posterior aspect of the spine in order to access the transverse processes. With an anterior, anterolateral, or lateral surgical approach, the surgeon must navigate through the abdominal cavity or retroperitoneal space or the chest cavity, placing many anatomic structures at risk. Described herein is an alternative fusion system and technique that employs a posterior approach to the spine, but avoids navigating past the neural elements, and avoids extensive muscle stripping. This percutaneous spinal fusion technique does not require access to the disk space, instead relying on fusion of the facet joints, and involves less-invasive muscle splitting rather than muscle stripping. This described technique allows for a posterior facet fusion through the same incision(s) used to place percutaneous pedicle screws, without accessing the interbody space, thus minimizing surgical trauma to the patient.

Described herein are a system and technique to perform a facet fusion through small incisions, allowing for a minimally invasive facet fusion. The main benefits of this technique are the simplicity and low risk, because it does not require accessing the disk space with the associated issues navigating past the neural elements. Rather, the system and technique described herein involves muscle splitting rather than muscle stripping, and is performed through the same small incisions that are routinely placed during percutaneous pedicle screw instrumentation. The technique is primarily designed to be used with the described minimally invasive percutaneous approach, but can also be employed in more conventional open surgical techniques. It should be understood that the terminology used in this present disclosure is for the purpose of description by way of example and is not intended to be limiting in any way.

To perform the percutaneous facet fusion, an incision is made through the skin, subcutaneous tissues, and fascia in the standard size and fashion used to place a pedicle screw directly below the facet to be fused. The percutaneous pedicle screw incision is placed in a manner well known to those schooled in the art. The percutaneous pedicle screw incision is usually approximately 1 centimeter (cm) in length, but can be shorter or longer. When a percutaneous facet fusion is performed, the fusion is generally performed prior to pedicle screw placement, but could be performed after pedicle screw placement if the incision was extended. After making an incision in a location and manner well known to those schooled in the art of percutaneous pedicle screw placement, blunt dissection is performed down to the facet joint, and a drill guide of approximately 5-10 millimeters (mm) in diameter is placed through the incision and docked onto the facet joint to be fused. This can be done freehand, or the drill guide can be passed along a guide wire as later described, and may be placed with fluoroscopic or other imaging guidance.

In some embodiments, the drill guide is placed freehand, without using a guide wire. In this embodiment, a blunt inner cannula may be placed in the drill guide to facilitate blunt dissection down to the facet joint, or the drill guide can be placed without the inner cannula. Once the drill guide is appropriately positioned on the facet, and the inner cannula removed if used, the drill bit is advanced through the drill guide approximately 5-8 mm into the facet joint. The drill bit depth can be determined based on pre-operative imaging and optionally on intra-operative imaging. The drill bit is then removed and bone graft or other bone growth promoting material placed through the drill guide with a tamp or other implement into the cylindrical void left by the drill bit. The bone graft or bone growth promoting material can be non-structural as in the case of an allograft bone putty or bone graft fragments, or can be more structural in nature as in the case of a pre-fabricated allograft that would be press-fit into the void. Once the bone graft or other bone growth promoting material is placed in the void, the drill guide is removed, and the percutaneous pedicle screw is then placed in the standard fashion through the same incision, in a manner well known to those schooled in the art. Because the facet joint is adjacent to the pedicle screw starting point, the elasticity of the skin and the soft tissues allows for translation of the incision cephalad and caudal a sufficient distance to allow both the facet fusion and percutaneous pedicle screw placement to be performed through the same incision.

In some embodiments, a percutaneous facet fusion technique involves passing the drill guide along a guide wire that has already been placed into the pedicle directly below the facet to be fused, as part of a percutaneous pedicle screw instrumentation system that relies on cannulated pedicle screws placed along a pedicle guide wire, in a manner well known to those schooled in the art. In some embodiments, an incision is made through the skin, subcutaneous tissues, and fascia in the standard size and fashion used to place the pedicle screw directly caudal to the facet to be fused. The fusion is performed first, followed by placement of the pedicle screw. The pedicle is cannulated in the standard fashion with a needle or probe, and a guide wire is placed through the needle or probe into the pedicle, and the needle or probe removed, leaving the guide wire in place. This technique is well known to those schooled in the art. In this embodiment, a sheath is attached to the drill guide, and the sheath would be just large enough to fit over and slide along the pedicle guide wire, and then the drill guide is advanced along the guide wire and docked onto the facet joint. The facet joint is located immediately cephalad to the pedicle starting point, so a drill guide passed along the pedicle guide wire will dock onto the facet joint. A blunt inner cannula may be placed in the drill guide to facilitate blunt dissection down to the facet joint, or the drill guide can be placed without the inner cannula, but either way, blunt dissection is carried out down to the facet joint. This embodiment has a potential advantage to the freehand technique in that the facet to be fused is reliably located directly cephalad to the cannulated pedicle, thus the drill guide can be accurately docked onto the facet and will be held in position by the guide wire during the facet fusion process. Once the drill guide is appropriately positioned and docked on the facet, and the inner cannula removed if used, the drill bit is advanced approximately 5-8 mm into the facet joint, the drill bit depth determined based on pre-operative imaging and/or intra-operative imaging. The drill bit is then removed and bone graft or bone growth promoting material packed through the drill guide and into the cylindrical void left by the drill with a tamp or other implement. The bone graft or bone growth promoting material can be loose or can be pre-fabricated to fit precisely into the void. Once the bone graft or other bone growth promoting material is placed in the void, the drill guide is removed, and the percutaneous pedicle screw is then placed in the standard fashion through the same incision, in a manner well known to those schooled in the art of percutaneous pedicle screw placement. Because the facet joint is adjacent to the pedicle screw starting point, the elasticity of the skin and the soft tissues allows for translation of the incision cephalad and caudal a sufficient distance to allow both the facet fusion and percutaneous pedicle screw placement to be performed through the same incision.

In some embodiments, the facet fusion can be performed during more conventional open surgery, rather than percutaneously. In this embodiment, the facet is exposed in the standard fashion through an open posterior extensile approach, and the drill guide docked onto the facet under direct visualization. The facet fusion then proceeds in the above described fashion, first drilling into the facet joint and then placing the bone graft or bone growth promoting material into the cylindrical void left by the drill bit, then optionally placing a pedicle screw as part of a spinal instrumentation construct.

The facet fusion may be performed unilaterally or bilaterally, and may be performed with or without spinal instrumentation, though it is designed primarily to be performed bilaterally at the spinal level that will be subsequently stabilized with spinal instrumentation.

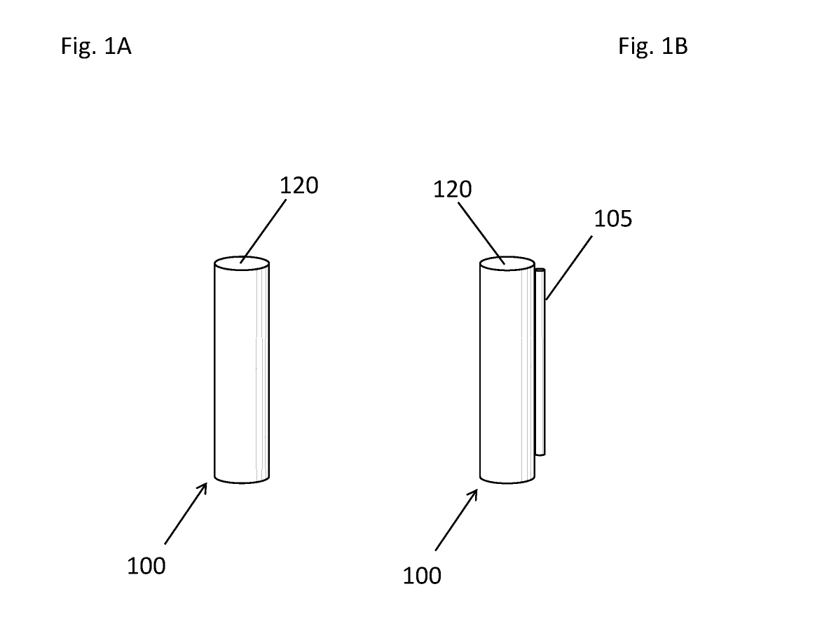

FIG. 1A depicts a drill guide 100 in accordance with an illustrative embodiment. In some embodiments, the drill guide is a cylinder with a central hole 120, through which a drill bit and/or bone graft or a bone growth promoting material can be passed. FIG. 1B depicts the drill guide 100 with the central hole 120, along with an attached sheath 105, in accordance with an illustrative embodiment.

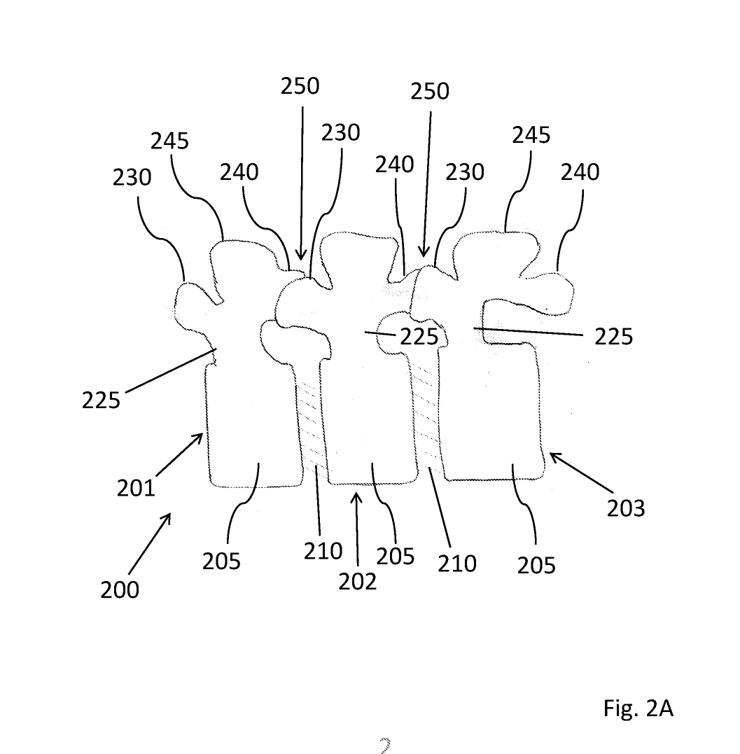

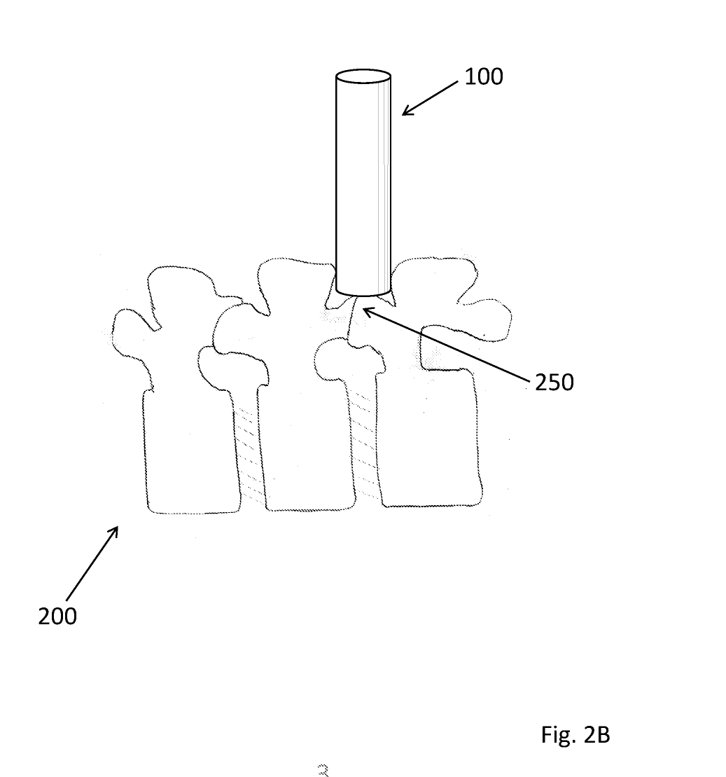

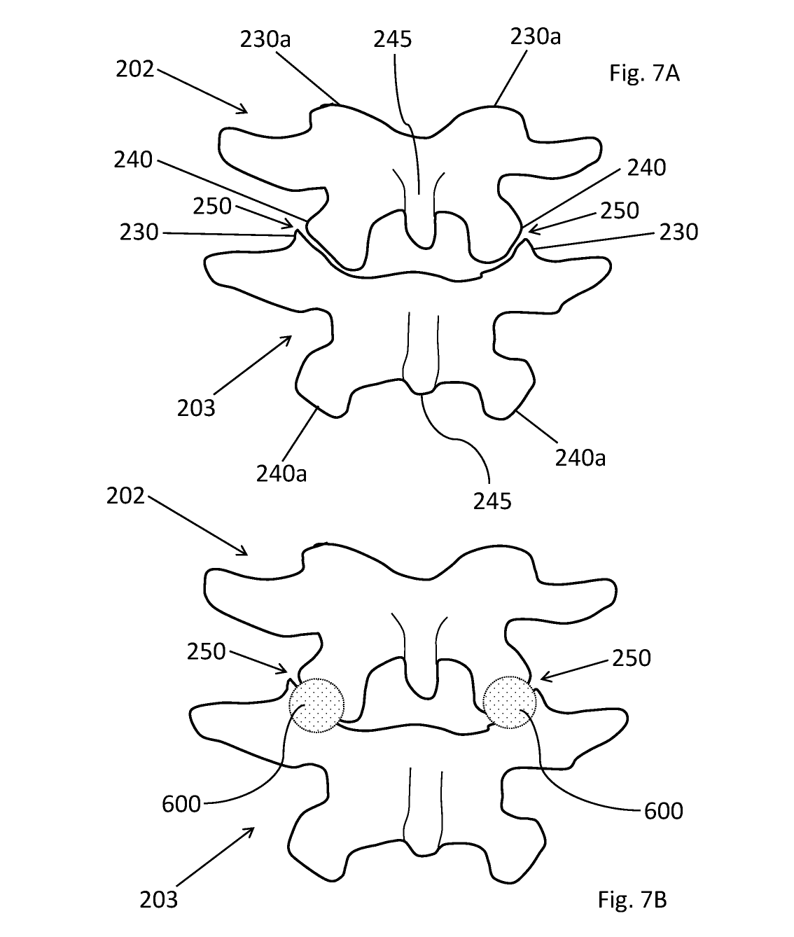

FIG. 2A depicts a lateral view of a lumbar spine segment 200, in accordance with an illustrative embodiment. Lumbar spine segment 200 consists of vertebrae 201, 202, and 203, with vertebra 201 the most cephalad and vertebra 203 the most caudal. Each of the vertebrae 201, 202, and 203 includes a vertebral body 205, bilateral pedicles 225 (although only the unilateral pedicle is seen in this lateral view), bilateral superior articular processes 230 (although only the unilateral superior articular process is seen in this lateral view), bilateral inferior articular processes 240 (although only the unilateral inferior articular process is seen in this lateral view), and a spinous process 245. An intervertebral disk 210 is located between a cephalad and caudal bodies. A facet joint 250 is comprised of the articulation of an inferior articular process 240 with a superior articular process 230. The facet joints are bilateral, although only one side is seen in this lateral view. In this depiction, a single vertebral level or segment may be defined as comprising a cephalad vertebra 201 and a caudal vertebra 202 and an intercalated disk 210, or a cephalad vertebra 202 and a caudal vertebra 203 and an intercalated disk 210. It should be noted that the terms “cephalad” and “caudal” with respect to an individual vertebra are relative terms, that is, a vertebra may be referred to as cephalad in one vertebral segment and caudal in another vertebral segment, or referred to as caudal in one vertebral segment and cephalad in another vertebral segment. In any given vertebral segment, the vertebra that is located closest to the patient's head is referred to as the cephalad vertebra, and the vertebra that is located furthest from the patient's head is referred to as the caudal vertebra. Thereby a cephalad vertebral body has bilateral inferior articular processes 240 (although only the unilateral inferior articular process 240 is seen in this lateral view) that articulate with bilateral superior articular processes 230 (although only the unilateral superior articular process 230 is seen in this lateral view) of a caudal vertebral body. The articulating relationship of articular processes 230 and 240 collectively forms bilateral facet joints 250 (although only the unilateral facet joint 250 is seen in this lateral view). This depiction therefore shows two vertebral levels or segments, one segment formed by vertebrae 201 and 202, the other segment formed by vertebrae 202 and 203, each segment having bilateral left and right facet joints 250 (though in this lateral spine depiction, only a unilateral facet joint 250 is shown; FIG. 7A and FIG. 7B depict bilateral facet joints).

FIG. 2B depicts a side view of drill guide 100 and a lateral view of the lumbar spine segment 200, in accordance with illustrative embodiments. After an incision is made and blunt dissection performed down to facet joint 250, drill guide 100 is docked onto the lumbar spine facet joint 250 using tactile feedback and/or intra-operative imaging.

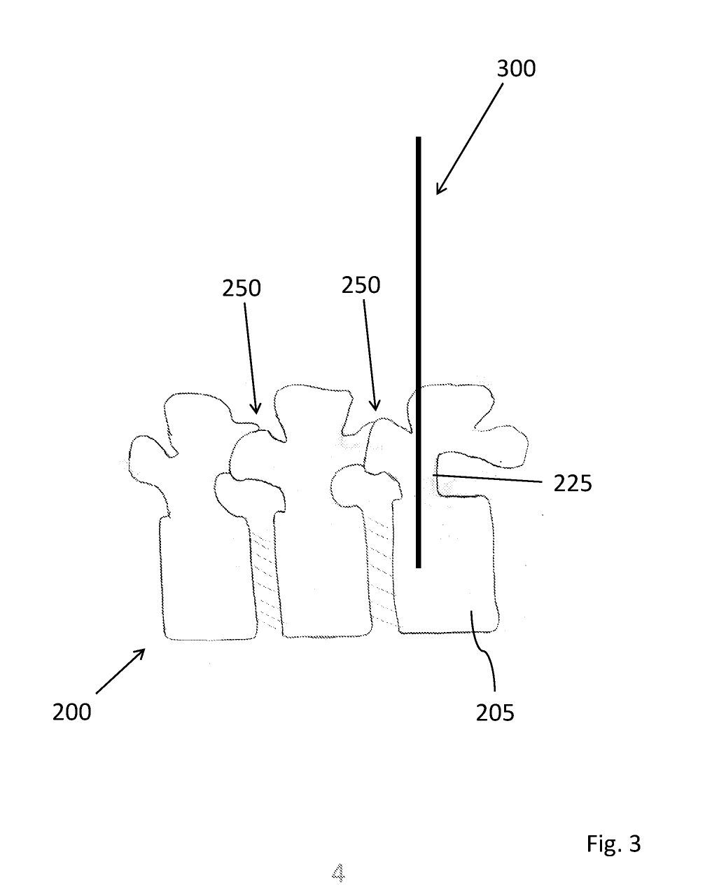

FIG. 3 depicts a lateral view of the lumbar spine segment 200, including facet joints 250, and a percutaneous pedicle screw guide wire 300, in accordance with illustrative embodiments. In some embodiments, percutaneous guide wire 300 has been placed through pedicle 225 into vertebral body 205 in anticipation of placing a percutaneous cannulated pedicle screw in a manner well known to those schooled in the art. Prior to placing a cannulated percutaneous pedicle screw along the guide wire, the guide wire is used to guide the drill guide 100 (not shown) onto the facet joint 250 in anticipation of performing the percutaneous facet fusion.

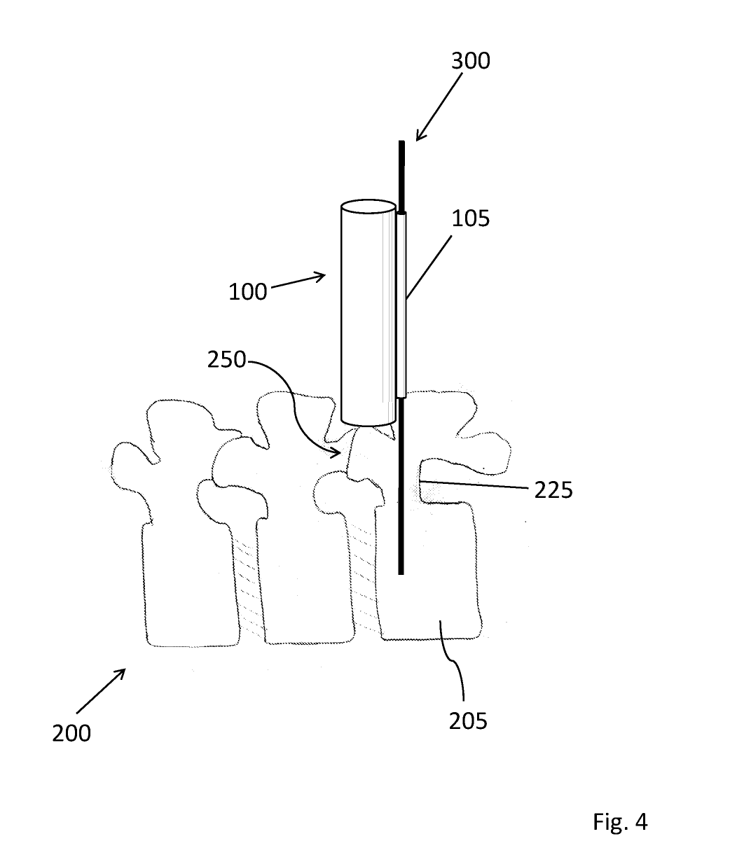

FIG. 4 depicts a side view of the drill guide 100 and attached sheath 105, and a lateral view of the lumbar spine segment 200, in accordance with illustrative embodiments. In one embodiment, the sheath 105 is detachably mounted to the drill guide 100 such that the sheath 105 can removed in situations when a guide wire is not used. Alternatively, the sheath 105 may be permanently mounted to the drill guide 100. The drill guide 100 is passed along the guide wire 300, with the guide wire 300 contained within the sheath 105, and drill guide 100 is docked onto the facet joint 250 in anticipation of performing the percutaneous facet fusion. Tactile feedback and/or intra-operative imaging may be used to facilitate drill guide 100 placement into the proper position on the facet joint 250.

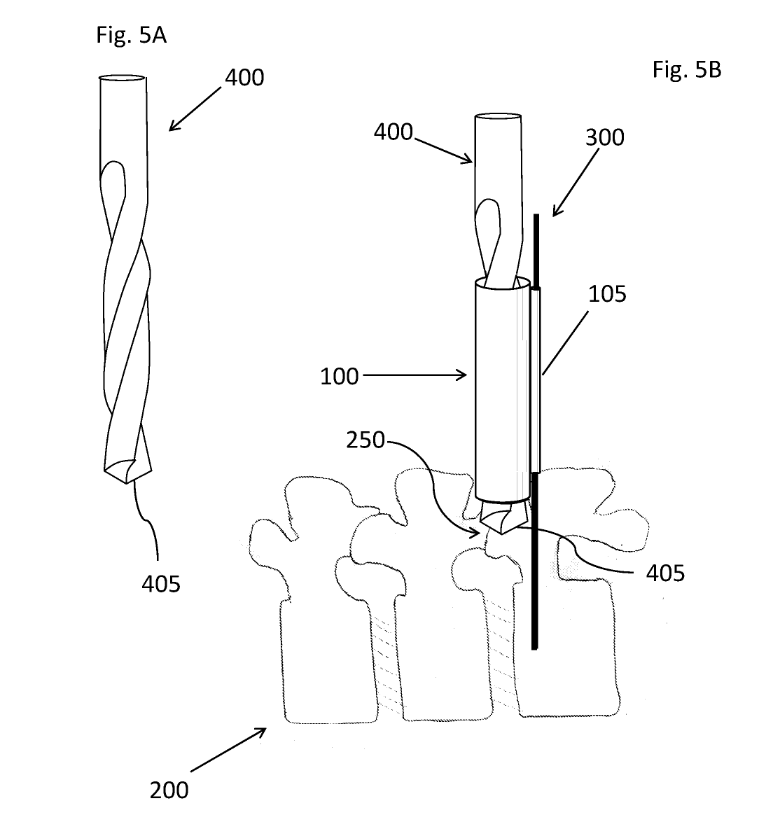

FIG. 5A depicts a drill bit 400 with a drill bit tip 405 that in FIG. 5B is depicted passing through drill guide 100 with attached sheath 105 that passes along pedicle guide wire 300, in accordance with illustrative embodiments. A lateral view of lumbar spine segment 200 is shown. Drill bit tip 405 can be drilled into facet joint 250, which will create a void for placement of bone graft and/or bone growth promoting material. In some embodiments, drill bit 400 is advanced approximately 5-8 mm into the facet joint, the drill depth based on pre-operative and/or intra-operative imaging and/or tactile feedback during surgery.

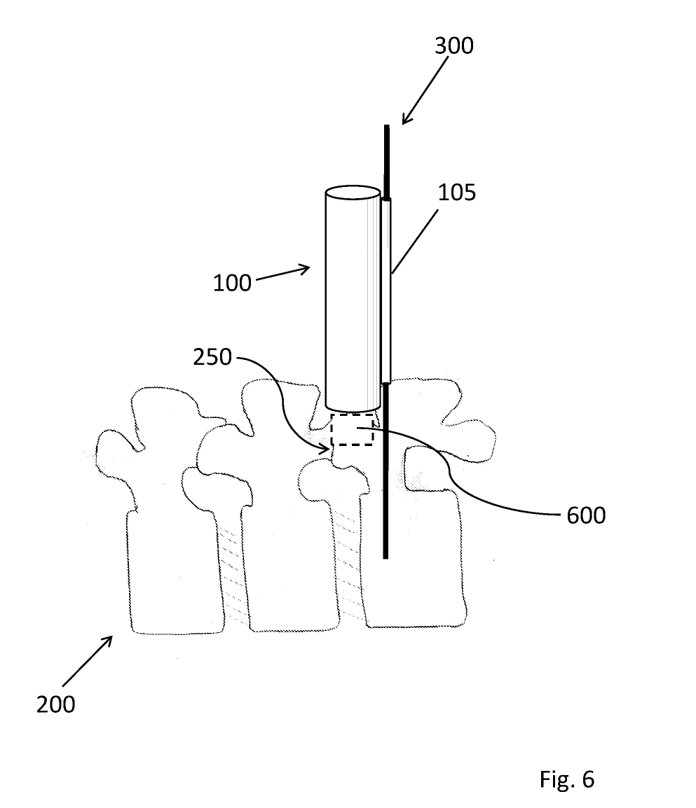

FIG. 6 depicts lumbar spine segment 200 and the drill guide 100 with attached sheath 105 that passes along pedicle guide wire 300, and drill bit 400 (not shown) has been removed from drill guide 100, in accordance with illustrative embodiments. The drill bit has created a void 600 in the facet joint 250. Referencing FIG. 2A, facet joint 250 is comprised of the articulation of inferior articular process 240 with superior articular process 230. The drill removes part of the inferior articular process and part of the superior articular process, thus leaving a void in facet joint 250, with freshly drilled exposed bone surfaces on the inferior and superior articular processes. Bone graft or a bone growth promoting material may be passed through the drill guide 100 and delivered into the void 600. This will provide the biologic component of the fusion and promote new bone growth from the freshly drilled exposed bone of the inferior articular process of the cephalad vertebra to the freshly drilled exposed bone of the superior articular process of the caudal vertebra, thus producing a bony fusion across facet joint 250. After delivery of bone graft or a bone growth promoting material into void 600, drill guide 100 is then removed, and then the spinal instrumentation is completed in a manner known to those schooled in the art of percutaneous pedicle screw placement. In some embodiments, bone graft or a bone growth promoting material could be placed into void 600 after the drill guide 100 is removed.

FIG. 7A is a posterior view of a depiction of a vertebral segment consisting of, for example, cephalad vertebra 202 and caudal vertebra 203, in accordance with an illustrative embodiment. Inferior articular processes 240 of cephalad vertebra 202 articulate with the superior articular processes 230 of caudal vertebra 203, forming facet joints 250. Spinous processes 245 are also shown, as are the superior articular processes 230a of cephalad vertebra 202 and the inferior articular processes 240a of caudal vertebra 203, for anatomical orientation.

FIG. 7B is a posterior view of a depiction of a cephalad vertebra 202 and a caudal vertebra 203, with facet joints 250, as shown in FIG. 7A, in accordance with an illustrative embodiment. Now depicted are voids 600 that have been created by the drill bit (not shown) as described in FIG. 5B and FIG. 6. Bone graft or a bone growth promoting material is placed into void 600, in order to produce the biologic environment that is designed to produce a bony fusion across facet joints 250.

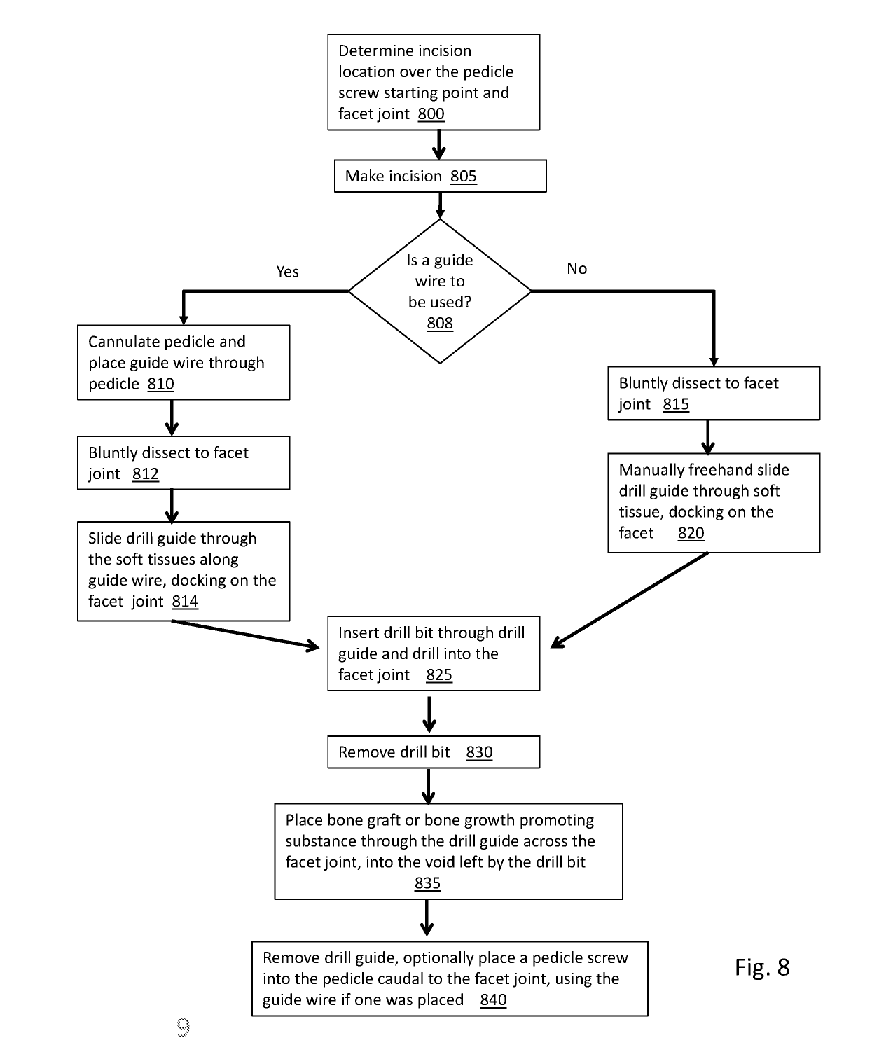

FIG. 8 is a flow diagram summarizing the operations of the percutaneous facet fusion technique. In alternative embodiments, fewer, additional, and/or different operations may be performed. Additionally, the use of a flow diagram is not meant to be limiting with respect to the order of operations performed. In an operation 800, the surgeon determines an incision location. The incision location may be determined with the use of fluoroscopy or other form of intra-operative imaging such as computed tomography, in a manner known to those with skill in the art of percutaneous pedicle screw placement. Alternatively, any other method(s) known to those of skill in the art may be used. In an operation 805, the surgeon makes an incision at the determined incision location. The surgeon then may proceed down one of two pathways based on a determination of whether a guide wire is to be used in an operation 808.

If it is determined that a guide wire is to be used in the operation 808, the surgeon cannulates the pedicle and places a percutaneous pedicle screw guide wire through the pedicle into the vertebral body in an operation 810. Cannulation of the pedicle and placement of the guide wire are done in accordance with techniques known to those of skill in the art. In an illustrative embodiment, the guide wire is placed below the facet being fused. In an operation 812, the surgeon bluntly dissects to the facet joint. In an operation 814, the surgeon slides a drill guide through the soft tissue and along the guide wire until the drill guide is docked on the facet joint. Specifically, the guide wire placed through the pedicle is placed into a sheath that is attached to the drill guide, and the drill guide is slid down the guide wire and docked onto the facet joint using tactile feedback and optionally intra-operative imaging to accurately place the drill guide on the facet. As such, the guide wire is used to direct the drill guide onto the facet joint.

Alternatively, the surgeon may decide to use a freehand technique that does not use a guide wire to direct the drill guide onto the facet joint. If it is determined that a guide wire is not to be used in the operation 808, the surgeon bluntly dissects to the facet joint in an operation 815, which is the same process that would be taken by the surgeon in the operation 812. In an operation 820, the drill guide (which may not have an attached sheath) is docked onto the facet joint in a freehand manner, using tactile feedback and optionally intra-operative imaging to accurately place the drill guide. The remaining operations are the same regardless of whether a guide wire is being used.

In an operation 825, the surgeon inserts a drill bit through the drill guide and the drill bit is advanced into the facet joint to form a cavity. The drill bit is removed in an operation 830. In an operation 835, a bone graft or bone growth promoting material is placed through the drill guide and into the void created by the drill bit in the facet joint. In an operation 840, the drill guide is removed and the remainder of the surgery performed in the standard fashion in a manner well known to those of skill in the art. If present, the pedicle guide wire can also be used to guide a cannulated percutaneous pedicle screw. If a guide wire is not used, the percutaneous pedicle screw can be placed without a guide wire, in a manner well known to those schooled in the art of guide wire-less percutaneous pedicle screw placement. In an alternative embodiment, the drill guide can be removed prior to placing the bone graft. These operations can also be performed in open surgery rather than percutaneous surgery, using any other method(s) known to those of skill in the art.

The components described herein can be made in a variety of lengths and/or shapes to accommodate various patient anatomies and surgeon preferences. The components can be made from stainless steel, plastic, titanium, titanium-alloy, cobalt-chrome, or any suitable material that is able to withstand the biomechanical stresses under which they will be placed.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A system for percutaneous facet joint fusion, the system comprising:
a guide wire configured for placement through an incision and into a pedicle of a first vertebra, wherein the incision is posterior to the first vertebra;
a drill guide with an opening that is configured to receive a drill bit;
a sheath mounted to the drill guide, wherein the sheath is configured to receive the guide wire such that the drill guide can be positioned adjacent to a facet joint formed by the first vertebra and a second vertebra; and
the drill bit, wherein the drill bit is configured to create a void in the facet joint by way of the drill guide, and wherein the void is configured to receive a bone graft or bone growth promoting material.

2. The system of claim 1, wherein the guide wire is a percutaneous pedicle screw guide wire.

3. The system of claim 1, further comprising the bone graft or bone growth promoting material.

4. The system of claim 3, wherein the bone graft or bone growth promoting material is configured for placement through the drill guide and into the void.

5. The system of claim 3, further comprising a tamp that is configured to fit within the drill guide such that the bone graft or bone growth promoting material can be forced into the void.

6. The system of claim 1, wherein the sheath is detachably mounted to the drill guide.

7. The system of claim 1, further comprising a needle or probe configured to cannulate the pedicle of the first vertebra.

8. The system of claim 1, further comprising a blunt inner cannula within the drill guide to facilitate blunt dissection down to the facet joint.

9. A method for performing percutaneous facet joint fusion, the method comprising:
inserting a guide wire through an incision and into a pedicle of a first vertebra;
inserting the guide wire into a sheath of a drill guide;
sliding the drill guide and the sheath along the guide wire to position the drill guide adjacent to a facet joint formed by the first vertebra and a second vertebra;
placing a drill bit through an opening in the drill guide and drilling into the facet joint to create a void; and
placing a bone graft or bone growth promoting material into the void.

10. The method of claim 9, further comprising forming the incision such that the incision is posterior to the first vertebra.

11. The method of claim 9, wherein the guide wire is a percutaneous pedicle screw guide wire.

12. The method of claim 9, further comprising forming a cannulation in the pedicle that is configured to receive the guide wire.

13. The method of claim 9, wherein placing the bone graft or bone growth promoting material into the void comprises forcing the bone graft or bone growth promoting material through the drill guide and into the void.

14. The method of claim 9, further comprising using a tamp to place the bone graft or bone growth promoting material through the drill guide and into the void.

15. The method of claim 9, wherein the sheath is detachable from the drill guide, and further comprising mounting the sheath to the drill guide.

16. The method of claim 9, further comprising inserting a blunt inner cannula into the drill guide to facilitate blunt dissection down to the facet joint.

17. The method of claim 9, further comprising inserting a pedicle screw into one of the first vertebra or the second vertebra using the incision through which the guide wire was placed.

18. A method for performing percutaneous facet joint fusion, the method comprising:
forming an incision that is posterior to a first vertebra;
inserting a drill guide through the incision;
positioning the drill guide such that the drill guide is adjacent to a facet joint formed by the first vertebra and a second vertebra;
placing a drill bit through an opening in the drill guide and drilling into the facet joint to create a void; and
placing a bone graft or bone growth promoting material into the void.

19. The method of claim 18, wherein placing the bone graft or bone growth promoting material into the void comprises forcing the bone graft or bone growth promoting material through the drill guide and into the void.

20. The method of claim 18, further comprising inserting a blunt inner cannula into the drill guide to facilitate blunt dissection down to the facet joint.
